(19) United States
(12) Patent Application Publication
(10) Pub. No.: US 2004/0009542 A1
(43) Pub. Date: Jan. 15, 2004

(54) Apparatus and method for detecting bacteria in blood products

(75) Inventors: Larry Joe Dumont, Arvada, CO (US); Marlene Adele Bainbridge, Golden, CO (US); Tay Goodrich, Denver, CO (US); Keith Manica, Lakewood, CO (US)

Correspondence Address: GAMBRO, INC, PATENT DEPARTMENT, 10810 W COLLINS AVE, LAKEWOOD, CO, 80215, US

(73) Assignee: Gambro, Inc.

(21) Appl. No.: 10/428,513

(22) Filed: May 2, 2003

Related U.S. Application Data
(60) Provisional application No. 60/377,754, filed on May 3, 2002.

(51) Int. Cl. G01N 33/554; G01N 33/569; A61M 5/178

(52) U.S. Cl. 435/7.32; 604/164.08

(57) ABSTRACT

An apparatus and method for penetrating a bacteria detection culture bottle in an aseptic manner. The apparatus includes the use of a sample bulb pre-connected to a product bag along with a sampler coupler including a needle or hollow spike. The apparatus also includes for the use of a sample bag or sample bulb and penetrating needle or hollow spike pre-connected or sterilely connected to a product container so that a sample may be taken for bacteria detection without contaminating the product in the product container.

### RELATED APPLICATIONS

This application claims the benefit of priority from Provisional Application No. 60/377,754, filed May 3, 2002.

### FIELD OF INVENTION

The present invention relates to the field of bacteria detection in collected body fluids, blood or blood component products. More specifically, the invention relates to an aseptic system for providing a sample of body fluids, blood or blood component to a bacteria detection culture bottle without contaminating the body fluid, blood or blood product to be transfused.

### BACKGROUND

Contamination of blood supplies with infectious microorganisms such as HIV, hepatitis and other viruses and bacteria presents a serious health hazard for those who must receive transfusions of whole blood or administration of various blood components such as platelets, red cells, blood plasma, Factor VIII, plasminogen, fibronectin, anti-thrombin III, cryoprecipitate, human plasma protein fraction, albumin, immune serum globulin, prothrombin complex, plasma growth hormones, and other components isolated from blood. Blood donor screening procedures may miss contaminants, and sterilization procedures which do not damage cellular blood components but effectively inactivate all infectious viruses, bacteria and other microorganisms have not heretofore been available. Thus, transfusion of blood and blood products into patients may introduce levels of contaminating bacteria. Wagner et al. (Clin. Microbiol. Rev. 7:290-302, 1994 and Goldman et al. (Trans. Med Revs. 5: 73-83, 1991) have identified a number of different species of bacteria in blood transfused to patients who later developed septicemia.

At present, there are culture-based systems for determining if contaminating bacteria is present in blood, body fluids, and platelets. Such culture methods typically provide a sample of the collected product to a culture medium containing nutrients. Any bacteria therein will grow in the culture medium and any microorganisms therein may be detected.

One such culture medium is present in the BacT/Alert® culture bottle manufactured by bioMérieux Industry, Inc. The BacT/Alert detection system typically uses bottle having a pierceable top with a calorimetric sensor contained in the bottle. Reflected light is used to monitor the presence and production of carbon dioxide (CO₂) in the culture medium. If microorganisms are present in the blood, blood component or fluid sample provided to the culture bottle, the bacteria present will metabolize the nutrients and produce CO₂. The color of the color sensor changes with the production of carbon dioxide reflecting the presence of the microorganisms.

The BacT/Alert culture bottles are sterile and initially the pierceable top is covered with a plastic cap. After removal of the cap the top is disinfected with a substance such as alcohol. The sample of blood, blood component or other fluid product to be tested is inserted into the bottle through the top.

In the past, various methods have been used to collect blood or blood components. One such method is the separation of the desired blood components from donor blood by apheresis with return of the uncollected components to the donor. One type of apheresis system is shown in U.S. Pat. No. 5,653,887, and another apheresis system having a different type of separation vessel is shown in U.S. Pat. No. 6,354,986. In each system, the desired component is collected in a product bag such as the product or collection bags of U.S. Pat. No. 5,653,887.

In the past, a representative sample of the collected blood component product has been obtained from the product or collection bags of an apheresis procedure if desired. One protocol for collecting a sample used with the Gambro® Trima® Automated Blood Component Collection System uses a platelet product sampler including a squeezable bulb while another protocol uses tubing segments for sampling.

One disadvantage of bacteria detection using a culture bottle is that contamination can occur from the transfer of the sample to the bottle thus indicating a contaminated product that might otherwise be free of microorganism. Thus it can be seen that aseptic connection of a sampler or sample container to a culture bottle for bacteria detection is highly desirable. Another disadvantage of bacteria detection in a sample is that the product and sample could be contaminated during the transfer of the sample from the product bag to the sampler or sample container.

### SUMMARY OF THE INVENTION

The present invention relates to providing an aseptic connection between a blood sample container, bag, or sampler and a bacteria culture bottle or other container. In one embodiment, the invention uses a sample bulb or sampler pre-connected to a product bag along with a sampler coupler including a needle or hollow spike for penetrating a bacteria detection culture bottle in an aseptic manner.

Also contemplated is the use of a sample bag or sample bulb and penetrating needle or hollow spike pre-connected or sterilely connected to a product container so that a sample may be taken for bacteria detection without contaminating the product in the product container. Another embodiment of the invention is directed to the use of a sterile connection device to connect a sample container to a needle for insertion into a bacteria detection culture bottle.

The method of collecting a sample of a blood product for bacteria detection without contaminating the final product bag or container is further contemplated by the present invention.

Although the invention is described with respect to bacteria detection it is noted that the teachings of the invention could also be used with respect to virus detection, parasite detection or other microorganism detection. Further, the sampling techniques of the invention could be applied to detecting bacteria and microorganisms in cell cultures and tissue cultures.

### BRIEF DESCRIPTION OF THE DRAWINGS

FIG. 1 illustrates an extracorporeal tubing set or disposable for use in an apheresis system.

FIG. 2 is a detail top view of a product bag from the disposable of FIG. 1 with connected blood sampler.

FIG. 3 is a top plan view of a product bag and alternative sampler arrangement.

FIG. 4 is a top plan view of the sampler of FIGS. 1 and 2 sterile docked to a needle.

FIG. 5 is a top plan view of another alternative product bag and sampler arrangement with pre-connected needle.

FIG. 6 is a top plan detail view of the product bag and sampler similar to FIG. 5 with coupler arrangement.

FIG. 7 is a top plan view of a product bag with alternative sampler and coupler arrangement.

FIG. 8 is a top plan view of an alternative sampler and coupler to be aseptically connected to a culture bottle.

FIG. 9 is a top plan view of a sample attachment kit to be sterilely connected to a product bag or container.

FIG. 10 is a top plan view of an alternative sample attachment kit to be sterilely connected to a product bag or container.

FIG. 11 is an isometric view showing a needle attachment and bacteria detection culture bottle.

FIG. 12 is an isometric view showing a coupler and bacteria detection bottle.

FIG. 13 is a partially cross sectional view of the needle guard or coupler shown in FIG. 12 showing an interior adapter to adapt the guard or coupler for alternative bacteria detection devices.

### DETAILED DESCRIPTION OF THE INVENTION

The examples below are described with respect to a bacteria detection bottle. However, these descriptions are further intended to encompass other types of detection apparatus and containers.

One embodiment of the present invention relates to a disposable or tubing set for an apheresis system having a pre-connected sample bulb, sample container or sampler for providing a sample to a bacteria detection culture bottle. By pre-connection is meant connection prior to sterilization of the disposable or tubing set. Pre-connection of the sample bulb, sample container or sampler to the disposable or tubing set provides advantages in that it reduces the risk of bacteria contamination of the sample and the collected blood product. One embodiment described below further discloses a pre-connected apheresis disposable with both sample bulb, sample container or sampler and needle or hollow spike for insertion into a bacteria detection culture bottle. Although an apheresis disposable is disclosed, it is understood that the needle or hollow spike and sample bulb, sample container or sampler could also be pre-connected to any product bag including a whole blood collection bag. It is also understood that the needle or hollow spike and sample bulb, sample container or sampler can be pre-connected to a component collection bag that collects separated components from a whole blood separation container or vessel including a ring-type or annular-type separator. Also, it is understood that the sample bulb, sample container or sampler can be connected to the needle or hollow spike through a sterile connection after sterilization as described with respect to an additional embodiment below.

Although the invention is described with respect to blood and blood components it is understood that the invention can also relate to bacteria detection in other body fluids. Also, the invention can relate to bacteria or microorganism detection in tissue cultures and cell cultures. Further, the invention can relate to sampling techniques used for other purposes such as a complete blood count.

One embodiment relates to use with a bacteria detection culture bottle, although the present invention also has applicability with other bacteria detection devices and other types of culture containers. The invention further can be used whenever there is a need to take a sample of blood or blood product or other fluids or cultures without contaminating the blood, blood product or fluid or culture in the primary container. One advantage of the instant invention is that the sample can be collected and the product bag disconnected prior to insertion of the sample into a culture container. This allows isolation of the collected product for re-infusion and prevents additional potential contamination of such product through the bacteria detection process.

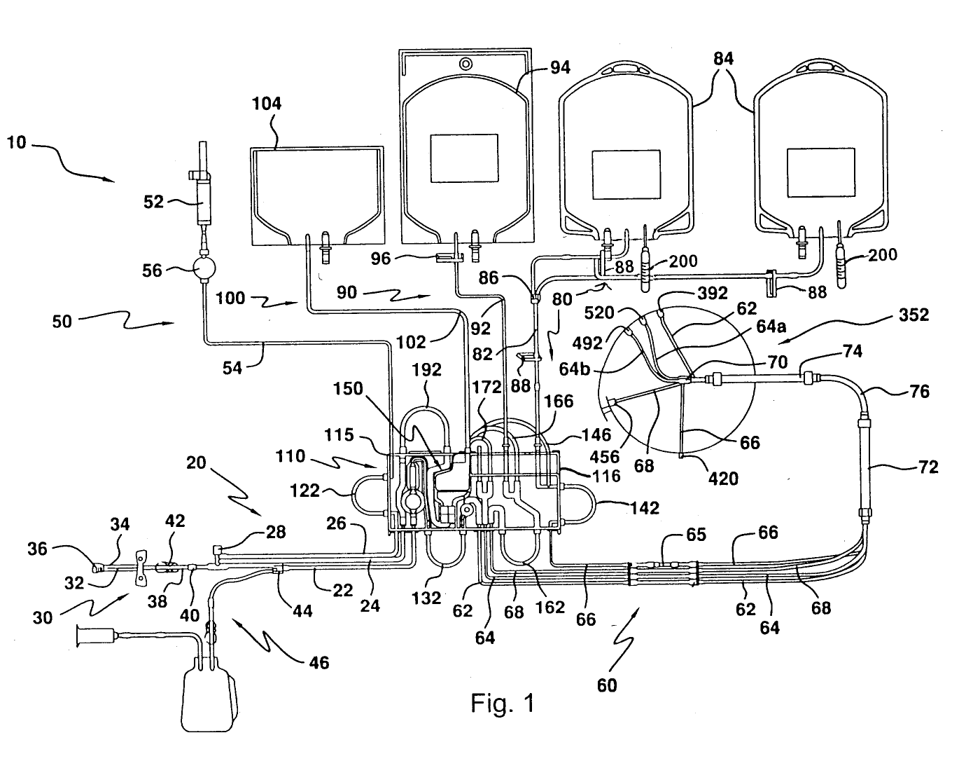

FIG. 1 is a disposable set of an apheresis system with an attached pre-connected sample bulb, sample container, or sampler 200 attached to product bags 84 in accordance with the present invention.

As illustrated in FIG. 1 extracorporeal tubing circuit 10 comprises a cassette assembly 110 and a number of tubing assemblies 20, 50, 60, 80, 90, 100 interconnected therewith. Generally, blood removal/return tubing assembly 20 provides a single needle interface between a donor/patient and cassette assembly 110, and blood inlet/blood component tubing subassembly 60 provides the interface between cassette assembly 110 and blood processing vessel 352. An anticoagulant tubing assembly 50, platelet collection tubing assembly 80, plasma collection tubing assembly 90, and vent bag tubing subassembly 100 are also interconnected with cassette assembly 110. As will be appreciated, the extracorporeal tubing circuit 10 and blood processing vessel 352 are interconnected to combinatively yield a closed disposable for a single use.

The blood removal/return tubing assembly 20 includes a needle subassembly 30 interconnected with blood removal tubing 22, blood return tubing 24 and anticoagulant tubing 26 via a common manifold 28. The needle subassembly 30 includes a needle 32 having a protective needle sleeve 34 and needle cap 36, and interconnect tubing 38 between needle 32 and manifold 28. Needle subassembly 30 further includes a tubing clamp 42 positioned about the interconnect tubing 38. Blood removal tubing 22 may be provided with a Y-connector 44 interconnected with a blood sampling subassembly 46.

Cassette assembly 110 includes front and back molded plastic plates that are hot-welded together to define a rectangular cassette member 115 having integral fluid passageways. The cassette assembly 110 further includes a number of outwardly extending tubing loops 122, 132, 142, 162, 192 interconnecting various integral passageways. The integral passageways are also interconnected to the various tubing assemblies.

Specifically, cassette assembly 110 includes a first integral anticoagulant passageway interconnected with the anticoagulant tubing 26 of the blood removal/return tubing assembly 20. More details of the internal passageways of cassette assembly 110 can be found in U.S. Pat. No. 5,653,887. The cassette assembly 110 further includes a second integral anticoagulant passageway and a pump-engaging, anticoagulant tubing loop 122 between the first and second integral anticoagulant passageways. The second integral anticoagulant passageway is interconnected with anticoagulant tubing assembly 50. The anticoagulant tubing assembly 50 includes a spike drip chamber 52 connectable to an anticoagulant source, anticoagulant feed tubing 54 and a sterilizing filter 56. During use, the anticoagulant tubing assembly 50 supplies anticoagulant to the blood removed from a donor/patient to reduce or prevent any clotting in the extracorporeal tubing circuit 10.

Cassette assembly 110 also includes a first integral blood inlet passageway interconnected with blood removal tubing 22 of the blood removal/return tubing assembly 20. The cassette assembly 110 further includes a second integral blood inlet,passageway and a pump-engaging, blood inlet tubing loop 132 between the first and second integral blood inlet passageways. The second integral blood inlet passageway is interconnected with blood inlet tubing 62 of the blood inlet/blood component tubing assembly 60.

Blood inlet tubing 62 is also interconnected with blood processing vessel 352 to provide whole blood through inlet port 392 thereto for processing. To return separated blood components to cassette assembly 110, the blood inlet/blood component tubing assembly 60 further includes red blood cell (RBC)/plasma outlet tubing 64, platelet outlet tubing 66 and plasma outlet tubing 68 interconnected with corresponding outlet ports 492 and 520, 456, and 420 of blood processing vessel 352. The RBC/plasma outlet tubing 64 includes a Y-connector 70 to interconnect tubing spurs 64a and 64b. The blood inlet tubing 62, RBC/plasma outlet tubing plasma outlet tubing 68 and platelet outlet tubing 66 all pass through first and second strain relief members 72 and 74 and a braided bearing member 76 there between. This advantageously allows for a sealless interconnection, as taught in U.S. Pat. No. 4,425,112.

Platelet outlet tubing 66 of the blood input/blood component tubing assembly 60 may include a cuvette 65 for use in the detection of red blood cells (via an interfacing RBC spillover detector provided on the blood component separation device) and interconnects with a first integral platelet passageway of cassette assembly 110. As will be appreciated, a transparent member could alternatively be integrated into cassette assembly 110 in fluid communication with first integral platelet passageway to interface with an RBC spillover detector.

The cassette assembly 110 further includes a pump-engaging, platelet tubing loop 142 interconnecting the first integral platelet passageway and a second integral platelet passageway. The second integral platelet passageway includes at least a first spur interconnected with platelet collection tubing assembly 80.

The platelet collection tubing assembly 80 can receive separated platelets during operation and includes platelet collector tubing 82 and platelet collection bags 84 interconnected thereto via a Y-connector 86. Slide clamps 88 are provided on platelet collector tubing 82 although it is further understood that frangible connectors or other types of clamps could also be used. The sampler of one embodiment of the present invention is shown at 200 attached to bags 84 as will be more fully described below.

A second spur of the second integral platelet passageway is interconnected with platelet return tubing loop 146 of the cassette assembly 110 to return separated platelets to a donor/patient (e.g., upon detection of RBC spillover during platelet collection). For such purpose, platelet return tubing loop 146 is interconnected to the top of a blood return reservoir 150 integrally formed by the molded front and back plates of cassette member 115. One or more types of uncollected blood components, collectively referred to as return blood, will cyclically accumulate in and be removed from reservoir 150 during use. Back plate of the cassette member 115 also includes an integral frame corner 116 defining a window through a comer of cassette member 115. The frame comer 116 includes keyhole recesses for receiving and orienting the platelet collector tubing 82 and platelet return tubing loop 146 in a predetermined spaced relationship within window.

The plasma outlet tubing 68 of blood inlet/blood component tubing assembly 60 interconnects with a first integral plasma passageway of cassette assembly 110. Cassette assembly 110 further includes a pump-engaging, plasma tubing loop 162 interconnecting the first integral plasma passageway and a second integral plasma passageway. The second integral plasma passageway includes first and second spurs. The first spur is interconnected to the plasma collection tubing assembly 90.

The plasma collection tubing assembly 90 may be employed to collect plasma during use and includes plasma collector tubing 92 and plasma collection bag 94. A slide clamp 96 or a frangible connector (not shown) is provided on plasma collector tubing 92. A sample bulb, sample container or sampler 200 (not shown) may also be attached to plasma bag 94 if sampling of the plasma collection is desired.

The second spur of the second integral plasma passageway is interconnected to a plasma return tubing loop 166 to return plasma to a donor/patient. For such purpose, the plasma return tubing loop 166 is interconnected to the top of the blood return reservoir 150 of the cassette assembly 110. Again, keyhole recesses in the frame 116 of cassette assembly 110 are utilized to maintain the plasma collector tubing 92 and plasma return tubing loop 166 in a predetermined spaced relationship within the window.

The RBC/plasma outlet tubing 64 of the blood inlet/blood component tubing assembly 60 is interconnected with integral RBC/plasma passageway of cassette assembly 110. The integral RBC/plasma passageway includes first and second spurs, respectively. The first spur is interconnected with RBC/plasma return tubing loop 172 to return separated RBC/plasma to a donor/patient. For such purpose, the RBC/plasma return tubing loop 172 is interconnected to the top of blood return reservoir 150 of the cassette assembly 110. The second spur may be closed off, or may be connected with an RBC/plasma collection tubing assembly (not shown) for collecting RBC/plasma during use. The RBC/plasma return tubing loop 172 (and RBC/plasma collector tubing if provided) is maintained in a desired orientation within the window by keyhole recesses of the frame 116.

Vent bag tubing assembly 100 is also interconnected to the top of blood return reservoir 150 of cassette assembly 110. The vent bag tubing assembly 100 includes vent tubing 102 and a vent bag 104. During use, sterile air present since packaging within cassette assembly 110, and particularly within blood return reservoir 150, cyclically passes into and back out of vent tubing 102 and vent bag 104.

Vent bag 104 may be provided with a sterile, gas pressure-relief valve at a top end (not shown). Further, it should be noted that, as opposed to vent bag tubing assembly 100, additional integral passageways, integrated chambers and tubing loops could be included in cassette assembly 110 to perform the same functions as the vent bag tubing assembly 100.

The platelet return tubing loop 146, plasma return tubing loop 166 and RBC/plasma return tubing loop 172 are interconnected in a row to the top of blood return reservoir 150 so that the blood components returned thereby will flow down the inner walls of the blood return reservoir 150.

A first integral blood return passageway is interconnected to the outlet of blood return reservoir 150, and is further interconnected to a second integral blood return passageway via a pump-engaging, blood return tubing loop 192. The second integral blood return passageway is interconnected with the blood return tubing 24 of the blood removal/return tubing assembly 20 to return blood to the donor/patient via needle assembly 30.

The apheresis tubing set described above is only representative of a tubing circuit or disposable which can be used with the present invention. The tubing set of U.S. Pat. No. 6,354,986 which further includes a leuko-reduction device can also be used. It is further understood that the teachings of the present invention can be applied to any apheresis disposable and to any product or collection bag within such disposable. It further can be applied to collection systems wherein whole blood is collected into a collection bag and then later separated into component products. By way of further example, a sampler (and/or needle or hollow spike) as more fully described below can be attached to a red blood cell collection bag, a plasma collection or even a bag intended for blood product storage after collection. The sampler (and/or needle or hollow spike) can also be attached to the apheresis collection bags of various apheresis systems of different manufactures. It can further be attached to product bags containing other bodily fluids when bacteria detection is desired.

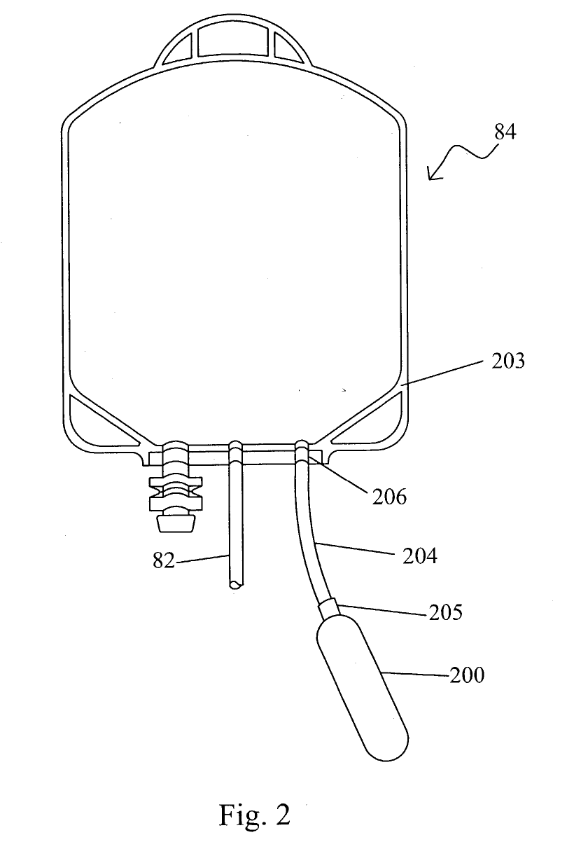

FIG. 2 is a detail view of the sampler 200 attached to the platelet collection bag 84. It is further understood such an attachment can be made to a whole blood collection bag or a blood product or blood component collection bag wherein the blood product can be collected by any known method other than by apheresis. Also, such an attachment can be made to a bag to which a blood product is transferred from the collection bag. Furthermore, the sampler can be attached to the red blood cell collection bag or the plasma collection bag as noted above.

Bags 84 and bags, 384, 400, 484 and 684 (described below) can be polymeric bags or other like containers. Such bags are typically constructed from one or two sheets of a polymeric material such as PVC or polyolefin, which may be welded together to form welds in the outer border zones 203 (FIG. 2). Such bags may also be made of tube type material with sealing or welding on only two sides.

The sampler 200 of FIG. 2 is shown attached to platelet collection bag 84. Platelet outlet line 82 is shown broken, but in the disposable 10 of FIG. 1 the line will interconnect through cassette assembly 110 to the platelet outlet tubing 66.

The sampler 200 of FIGS. 1 and 2 may be blow molded of polymeric material in a resilient sample bulb shape and adhered by bonding, welding or heat sealing, or other known methods at 205 to a first end of tubing 204. Bonding, welding or heat sealing may also be used to adhere a second end of the tubing 204 to the bag 84. For example, tubing 204 may be inserted into the outer border zone at 206 and sealed or welded into the border zone (FIG. 2). Alternatively, other known methods of attachment such as bonding can also be used. Also other forms of molding, such as injection molding, may be used for the sample bulb.

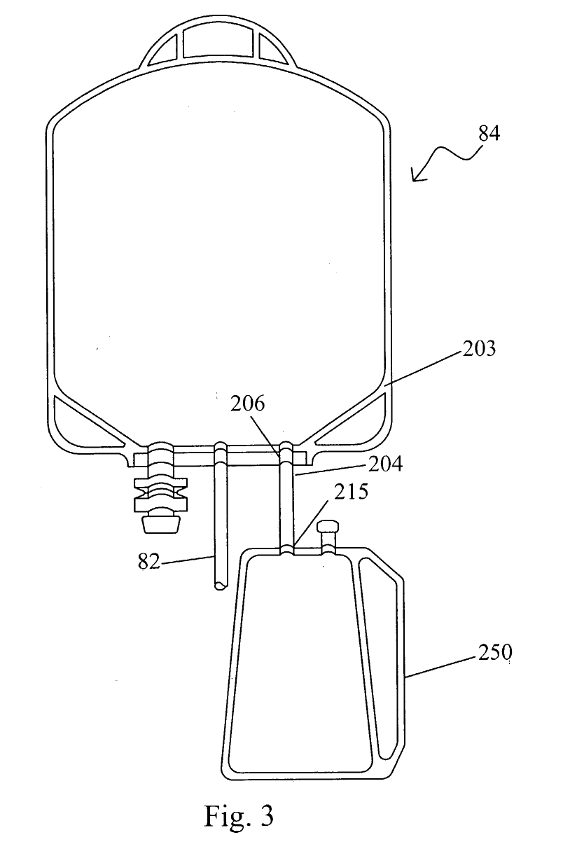

The sampler can also be a sample bag 250 as shown in FIG. 3 connected at 215 as described above with respect to the sample bulb. The other elements shown in FIG. 3 are the same as those of FIG. 2.

One advantage of pre-connection of at least the sample bulb, sampler or sample container 200, 250 to the blood or blood product collection or other bag is to provide a closed system for sample collection. Such a pre-connected disposable or tubing set may then be sterilized as a unit as noted above.

The operation of the pre-connected embodiment will now be described with specific reference to FIGS. 1 and 2, although it is also understood that the operation will also apply to the embodiment having the sample bag of FIG. 3. Also, although the operation will be described with respect to the platelet collection bag, it can be similarly used with respect to the plasma collection bag 94 or a red blood cell collection bag (not shown). Similar sample techniques can also be used with whole blood containers as well as containers for other bodily fluids as noted above. After collection of a blood or blood component product into the product bags 84 of FIGS. 1 and 2 as is fully described in U.S. Pat. No. 5,653,887, a blood or blood component sample may-then be collected by disconnecting the product bag 84 from the rest of the disposable set 10 after stripping the tubing 82 into bag 84. The bag 84 is generally allowed to rest a period of time before a sample is taken as is described below.

Alternatively, the sample may be taken immediately after collection of the collected product or may be taken anytime up to re-infusion of the collected product. Preferably the sample is taken twenty-four to forty-eight hours after collection, although this can be varied as noted above.

The contents of bag 84 are then mixed and then sample bulb 200 is lightly squeezed to fill the bulb with generally about 1.5 mL to 4 mL of platelet concentrate though these amounts may be varied. It is understood that this is a representative sample amount and not meant to be limiting and that the volume of the sample taken can, in particular, be larger if desired or needed for particular sampling purposes. Sample bulb 200 of FIGS. 1 and 2 can then be separated from the bag 84 by sealing the tubing 204 (in FIG. 2) in two places and then cutting the tubing 204 between the seal. Sealing of the tubing may be by heat or radio frequency energy or methods known in the art.

The process with respect to FIG. 3 is similar, and bag 250 can be filled by opening a frangible connector (not shown) in line 204 or a clamp to fill, through a gravity flow, the bag with between 1.5 ml to 4 ml or selected amounts of platelet concentrate from the product bag 84. As described above with respect to the sample bulb this can be done after product bag 84 is disconnected from any disposable set and line 82 is sealed.

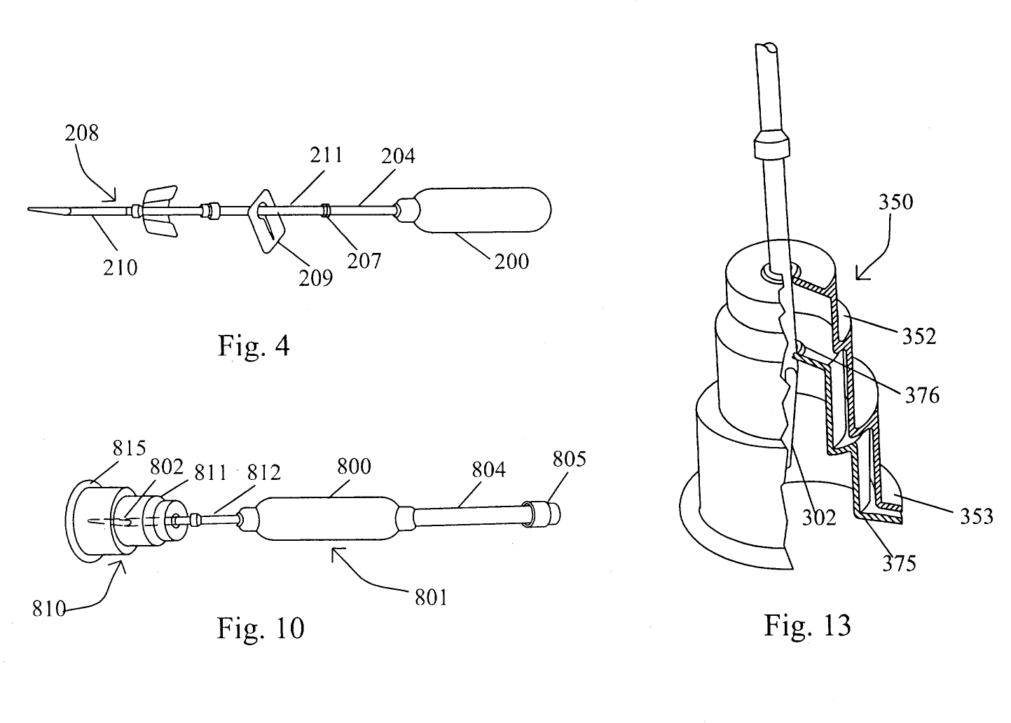

If a needle or hollow spike is not pre-connected to the disposable or tubing set, as is the case in FIG. 1, such needle or hollow spike may be connected using sterile docking techniques, as shown in FIG. 4. The sample bulb 200 is shown connected by a well-known sterile dock or connection technique to needle portion 208 having needle 210 in this figure. Needle portion 208 is pre-connected at a first end to tubing 211. A second end of tubing 211 is sealed using sterile connection techniques to tubing 204. The sterile connection is shown at 207. One connection device that can be used to provide a sterile connection or seal is the heat or sterile dock device shown in European Patent Application 0643975A1. Other known types of sterile connection devices could also be used. Sliding clamp 209 can be closed to prevent the sample fluid, blood or blood component from draining to the needle 210 until it is desired to begin the bacteria detection process as more fully described below. Alternatively slide clamp 209 can be used on the sample bulb side of the sterile connection if desired. Instead of a slide clamp a frangible connection or other type of known clamp such as a pinch clamp may be used to prevent fluid from leaving the sample bulb or bag prematurely. A similar needle or hollow spike can be sterilely connected to bag 250 if a bag is used instead of bulb 200 (not shown).

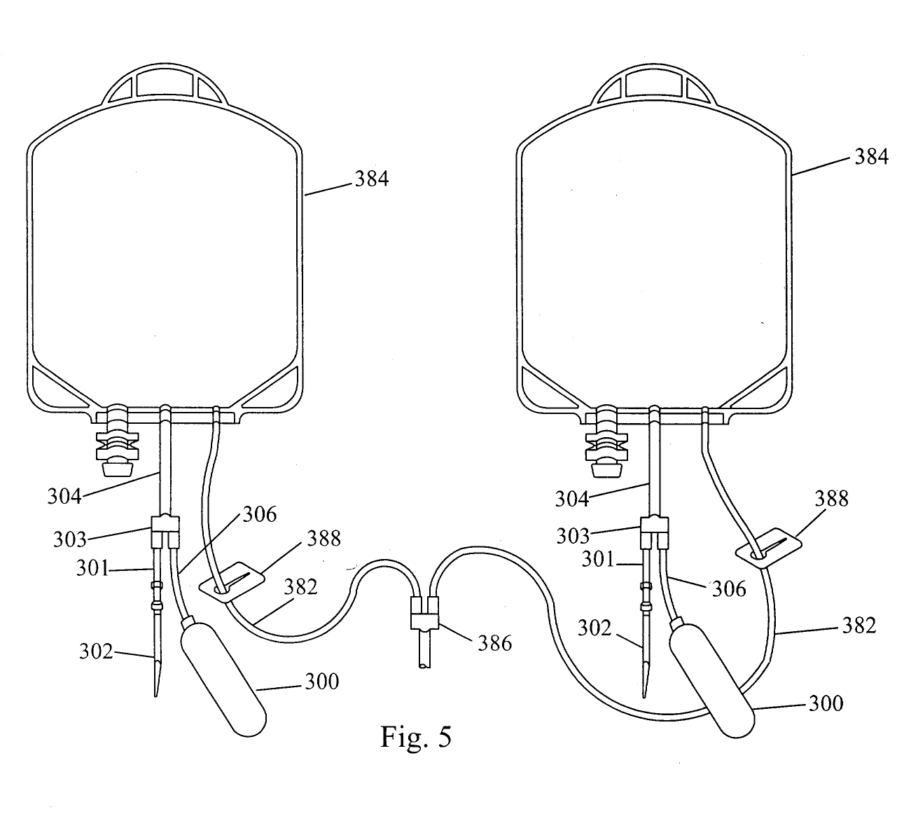

FIG. 5 illustrates a detail of a closed system disposable including pre-connected sample bulb 300 and pre-connected needle 302. Thus in this embodiment there is no need to sterilely connect a needle to a sampler or sample container. Bags 384 may correspond to collection bags of an apheresis system such as the bags 84 or 94 of FIG. 1 or they may be collection bags into which the fluid, blood, or blood component has been collected by other methods. It is also understood that the teaching of the present invention are applicable to a single collection or product bag although two are shown in FIG. 5. The various components will be described for one bag 384 below but it is understood that they are the same for the second bag 384.

Bag 384 of FIG. 5 has pre-attached or pre-connected tubing 304. Tubing 304 is pre-connected at a first end by heat sealing or welding or other methods to bag 384 as described above with respect to FIG. 2. Tubing 304 is pre-connected at a second end to Y-connector 303 although it is understood that other known connectors such as “T” or manifold connectors could be used. A tubing section 301 with needle or hollow spike 302 at a first end is pre-connected at a second end to the Y-connector again by known bonding, welding or sealing methods. Similarly tubing 306 with pre-connected sample bulb 300 at a first end is also pre-attached or pre-connected at a second end to the Y-connector. Although not shown a slide clamp or other type of clamp may be included on either 304, 306 or 301 if desired. Also a frangible connector or similar device could be used.

Similar to FIGS. 1 and 2, the sample bulb of the embodiment of FIG. 5 can be lightly squeezed to fill through Y-connector 303 and tubing 306 with a sample of the blood, fluid or blood components in bag 384. Tubing 304 may then be severed and sealed as close as practicable to Y-connector 303. The sample in sampler 300 can then be isolated from the product in bag 384. For bacteria detection the sample may flow through tubing 306, Y-connector 303, tubing 301 to needle or hollow spike 302 for use with a bacteria detection culture bottle as described below.

Tubing 382 corresponds to collection tubing 82 of FIG. 1 with slide clamp 388 and Y-connector 386 corresponding to elements 88 and 86, respectively. It is understood however that depending on the volume of the collected product the contents of one bag 384 may flow through the tubing 382 and Y-connector 386 to be mixed with the contents of the other bag 384 for sampling purposes. It is also understood that the contents of only one bag, (prior to mixing) may be sampled if desired. In this situation only one product bag will have a pre-connected sampler, Y-connection, and needle assembly. Tubing 382 may be severed and sealed as described above with respect to FIGS. 1 and 2 before any sample is taken.

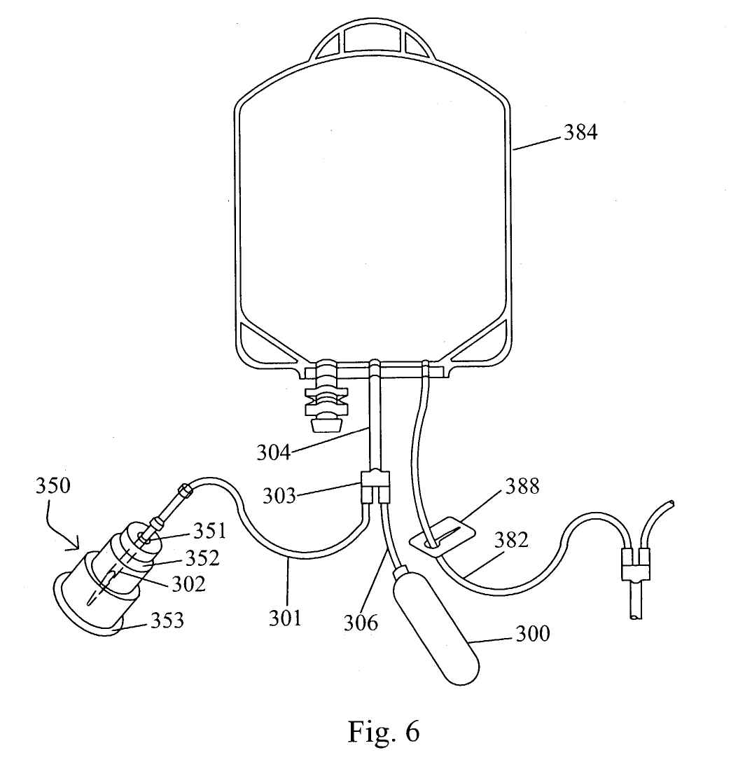

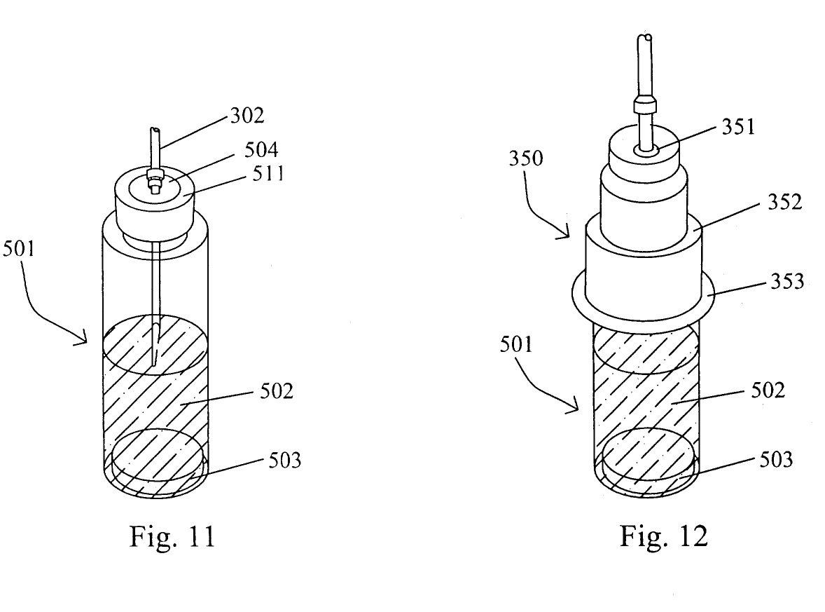

FIG. 6 is similar to FIG. 5 above with like reference numerals representing like elements (although only one product bag 384 is shown) except that the embodiment of FIG. 6 includes a sample site coupler 350 or guard connected around needle 302 connected to tubing 301 for aseptic connection to a bacteria detection bottle 501 (FIGS. 11 and 12) as more fully described below. The sample site coupler is connected at 351 around needle 302 and includes a shoulder 352 to accommodate the detection bottle 501, again, as described below. A flange 353 is also provided so that the coupler can be pushed onto the detection bottle using the flange 353 if desired. Although not shown, a slide clamp or other type of clamp may also be used on tubing 304, 301 and/or 306.

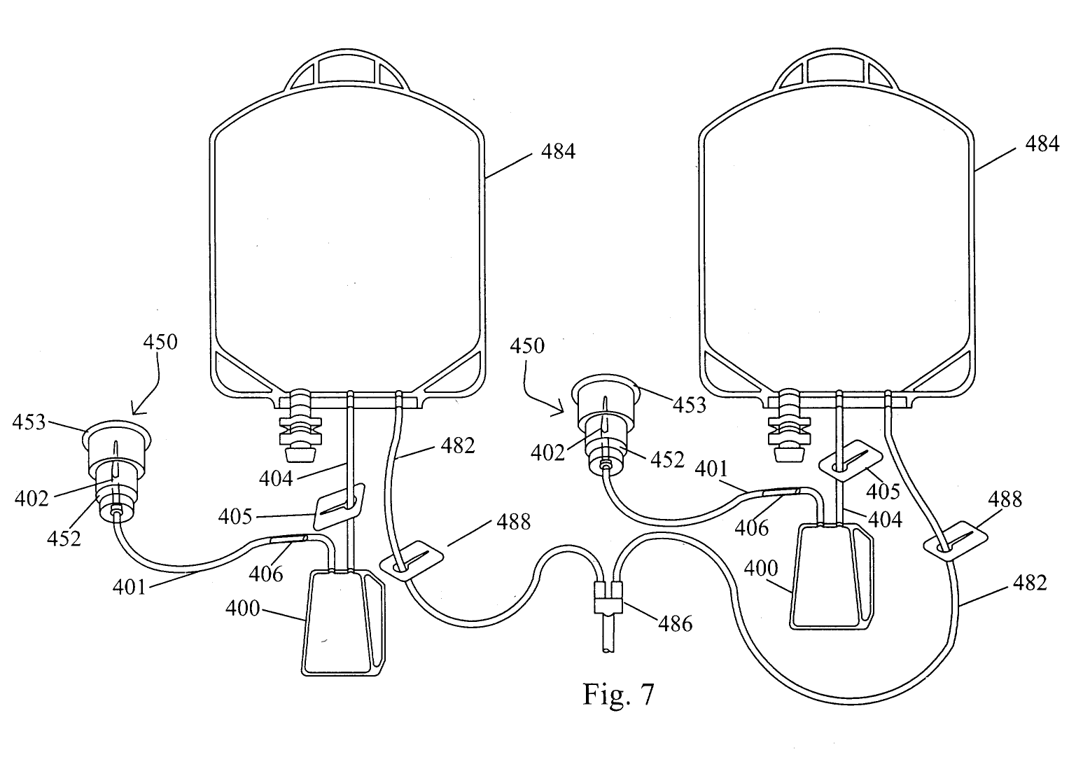

FIG. 7 is similar to FIG. 5 with collection bags 484, inlet tubing 482, slide clamps 488 and Y-connector 486. The sampling is achieved using a sample bag 400 rather than a sample bulb 300 as described below with respect to one of the collection or product bags 484. It is understood that the other sampling configuration for the second bag 484 is also similar. Again bags 484 may be the collection bags of an apheresis disposable or may contain blood, blood components or body fluids connected by other known methods. It is also understood that the teaching of this embodiment are applicable to a single product or collection bag. Again, if only a single sample is taken, only one product or collection bag will have the pre-connected Y-connector 486, sample bag 404, and sample site coupler 450. It is further understood that the product from one bag 484 may flow through tubing 482 and Y-connector 486 to the second product or collection bag 484 to mix with the product of the second bag 484. A sample may then be taken only from the bag 484 containing the mixed product. Also it is anticipated that a sample may optionally be taken of only one bag 484 even when both bags 484 contain product.

The sampling configuration will now be described more specifically with respect to FIG. 7. Tubing 404 is pre-connected at a first end by welding into the outer border zone of bag 484 as described above with respect to the previous embodiments. It is also understood that other methods can be used to pre-connect tubing 404 to bag 484 prior to sterilization. Tubing 404 has slide clamp 405 attached thereon although it is understood that another type of clamp or frangible connector could also be used. Sample bag 400 is pre-connected to a second end of tubing 404 by similar welding or sealing into the outer border zone of the bag 400 during manufacture. Alternatively tubing 404 could be bonded to bag 484 and/or bag 400. Tubing 401 is also pre-connected at a first end to bag 400 and at a second end to needle or hollow spike 402 using any known method as described above with respect to tubing 404. FIG. 7 is shown with a sample site coupler 450 similar to 350 of FIG. 6. If a configuration without such a coupler is preferred, the needle or coupler spike 402 can be used as shown in FIG. 5. The coupler or cover or guard 450 is secured over the needle or hollow spike 402 such as by screw threads, bonding, or other known methods. All parts described above are pre-connected prior to sterilization of the disposable or tubing set.

For use in sampling, slide clamp 405 is opened to drain the requisite sample into bag 400 from bag 484 through tubing 404. Two heat seals may be made in tubing 404 and the tubing may then be cut between such seals to isolate the product in bag 484. The sample contents of bag 400 may then be provided to a bacteria detection culture bottle as more fully described below through tubing 401 and needle 402. A frangible connector 406 or other clamp can be opened to allow the sample fluid to pass from bag 400 through tubing 401. Coupler cover or guard 450 fits over the culture bottle (not shown in FIG. 5) to assist in preventing the introduction of bacteria from a source external to the collection bags. The flange 453 and shoulder 452 assist in placing the coupler on a culture bottle to provide an aseptic connection.

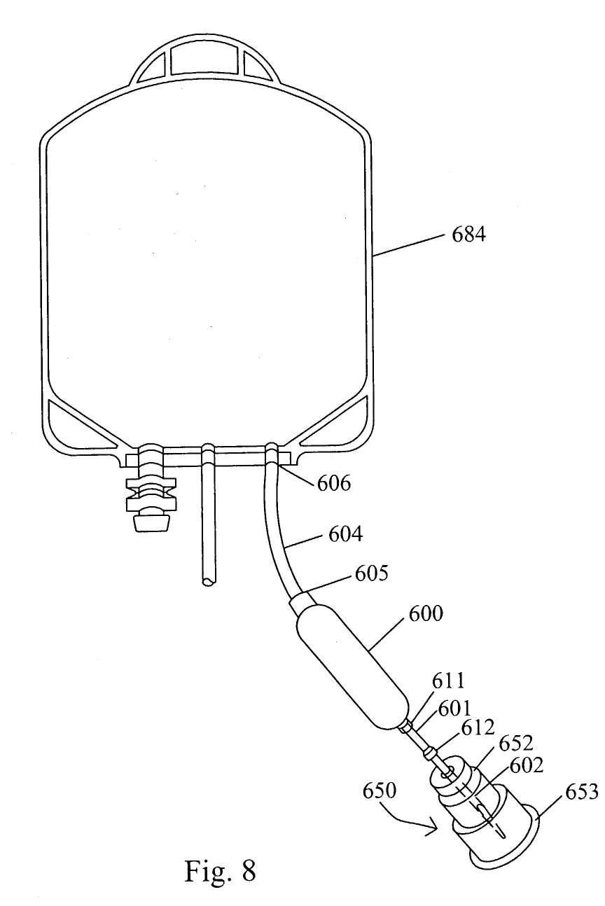

FIG. 8 illustrates an alternative sampler/needle/hollow spike with coupler arrangement. The coupler 650 in this figure is shown with a sample bulb rather than a sample container or bag but otherwise the operation can be similar to FIG. 7. This arrangement is shown only with one bag 684 but is understood the arrangement could be used for any number of bags in the disposable of FIG. 1. Also, the structure of FIG. 6 can be used when there is only one collection/product bag such as for a whole blood collection.

Bag 684, which is the product or collection bag, is pre-connected to a first end 606 of tubing 604 by known methods as described above with reference to the other embodiments. The second end 605 of tubing 604 is pre-connected to a squeezable sample bulb 600 at a first end also by known methods. The second end of sampler bulb is pre-connected to the first end 611 of tubing 601. The other 612 end of tubing 601 is pre-connected to needle or hollow spike 602 having coupler, cover or shield 650 which fits over a bacteria detection culture bottle and over the needle or hollow spike 602 as described further below.

In use bulb 600 may be lightly squeezed to remove the desired sample from bag 684 after bag 684 has been disconnected from a disposable set. Sample bulb 600 may be removed from bag 684 by heat sealing or other methods for processing. Slide clamps or other clamps (not shown) may also be included on tubing 601, 604 or a frangible clamp may be used. Needle 602 can then be inserted into a bacteria detection culture bottle with coupler 650 coupling with the bottle to provide the sample contents from bulb 600 as described below. The coupler 650 further includes shoulder 652 and flange 653, similar to the coupler previously described.

It is understood in all the embodiments above that all pre-connections can be made using known connection methods and that the pre-connections can be made prior to sterilization to provide a closed disposable. It is also understood that other well known types of clamps other than slide clamps can also be used.

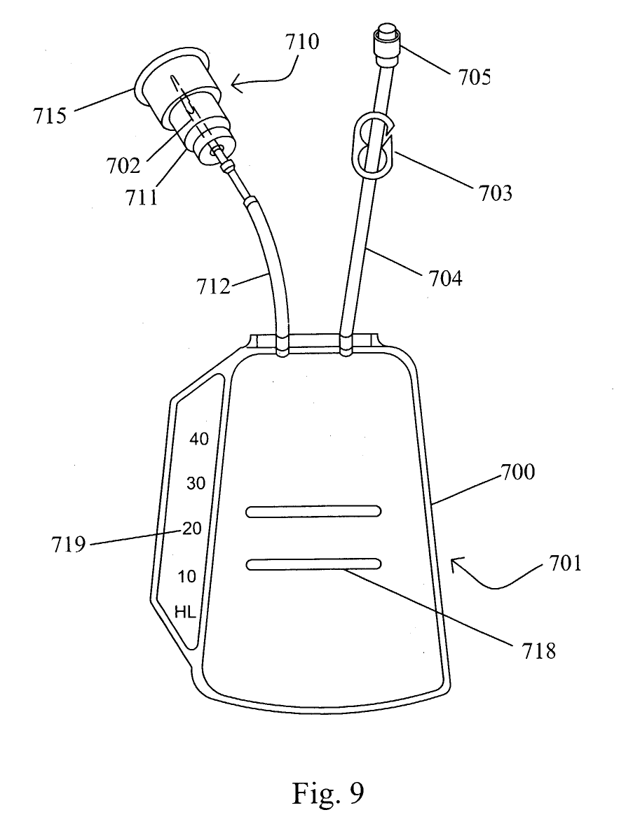

FIG. 9 discloses an alternative configuration wherein the sampler or sampling configuration is not pre-connected to a blood collection/separation disposable set. The sampling kit 701 of FIG. 7 is adapted to be sterilely connected to a tube such as 84 of a product bag such as 84 of a collection/separation disposable such as that of FIG. 1. Alternatively, this could contain other fluids or cultures to be sampled. The sampling kit or configuration 701 includes a sample bag 700 although another type of container including a sample bulb could also be used as described below with respect to FIG. 10. Tubing 712 and 704 is shown connected to sample bag by welding, bonding, heat sealing or other known methods. A protective cover 705 is placed on an end of tubing 712 opposite the end attached to the bag 701. A clamp 703 is also provided on tubing 704, although one could also be placed on tubing 712. A frangible connector or other type of flow-occluding-type device can also be used.

A needle 702 with sampler coupler 710 is attached to tubing 712 for cooperative connection to a bacteria detection container as more fully described below. The sampler coupler includes shoulder 711 and flange 715 for cooperating with the bacteria detection container as described below and for covering and protecting the needle 702. FIG. 9 also shows in detail markings 718 and numbers 719 on sample bag 700. All sample bags as well as sample bulbs or containers can have desired markings or desired indicia to indicate fluid levels or other information.

FIG. 10 is similar to FIG. 9 except that the sampling kit 801 includes a sample bulb 800 instead of the bag 700. Cap 805 is attached to tubing line 804 which at a second end is connected to bulb 800, all connections being by known methods as described with respect to FIG. 8. Tubing 812 is attached to bulb 800 at one end and to needle 802 at the other end. Sample coupler 810 includes shoulder 811 and flange 815 around needle 802 for cooperative coupling to a bacteria detection bottle as more fully described below.

To connect the sample kit 701 or the sample kit 801 to a collection bag the tubing 704 between the cap 705 and bag 700 or tubing 804 between the bulb 800 and the cap 805 is sterilely connected to tubing connected to a blood product or blood component or other bag for sampling (not shown). The portion of tubing 804 proximate to and including cap 805 and the portion of tubing 704 proximate to and including cap 705 can then be disposed of. After connection using sterile connection techniques as described above the set for sampling will be similar to that sampling portion of the disposable shown in FIG. 7 (bag) or similar to FIG. 8 (bulb). The sterile connection will preferably be made after the product or collection bag (for example 684 or 484) is removed from the rest of the disposable set. The sample can then be taken as described above.

The use of the samples collected into sample bulbs 200, 300 and 600, 800 and bags 250, 400 and 700 will now be described with reference to a bacteria detection culture bottle with reference to FIGS. 11 and 12. Bottle 501 includes culture medium 502 and a sensor such as a colormetric CO₂ sensor 503. For use a cap (not shown) is removed from the culture bottle top 511. A top surface portion 504 is adapted to be pierced by a needle or hollow spike illustrated as 302 from the embodiment of FIG. 5. It is also understood that any of the embodiments can be used with the culture bottle of FIGS. 11 and 12. Prior to piercing with needle or hollow spike 302 as shown in FIG. 1 the top surface 504 is cleaned by wiping with a sterile disinfectant wipe. During piercing any flexible cover, (if used), on the needle or hollow spike will be removed from the needle. After piercing, the collected sample is introduced from sample bulbs 300, or from the other bulbs or bags into bottle 501. Sensor 503 changes color upon production of CO₂. Growth of microorganisms in the sample produces the CO₂ or carbon dioxide to be detected. A detector for determining the color change in sensor 503 is provided as more fully explained in U.S. Pat. No. 5,164,796. It is understood that although the described bacteria detection culture bottle is specific for detection of the presence of CO₂ other characteristics of microorganism growth could also be detected including, but not limited to, depletion of oxygen. Therefore any bacteria sensor could be used.

FIG. 12 illustrates the same bacteria detection bottle for use with a coupler such as the coupler 350, 450, 650, 710 and 810 of FIGS. 6-10. Again, bottle 501 includes culture medium 502 and a sensor such as CO₂ sensor 503. For use, a cap (not shown) is removed and top surface 504 is cleaned as described above. The coupler (350 in this example, though it is understood the process will be the same for the other couplers) is pressed onto bottle 501 through the use of flange 353 if desired. Needle 302 (not shown in FIG. 12) pierces top surface 504, (removing any needle cover), as shoulder 352 rests on top surface 504 of the bottle 501. This provides aseptic communication between the bottle 501 and needle 302.

FIG. 13 illustrates an adapter for a coupler to allow the coupler to cooperate with various bacteria detection bottles or configurations. For purposes of this example coupler 350 with shoulder 352, flange 353 and needle 302 is shown with insert 375. Insert 375 can be in any shape to fit inside coupler 350 while being able to cooperate with a bacteria detection bottle or container. Insert or filler 375 fits around needle 302 such as shown at 376 and can have ridges or spacers, (not shown), to make it the proper shape for cooperation. Insert 375 can also be of flexible material so that it can flex into the coupler to best fit the shape of the bacteria detection bottle.

The teachings of the above invention can also be used with respect to syringes. That is, if a sample is taken with a syringe the syringe can have a coupler for guarding the needle to provide an aseptic communication with a culture container.

With respect to the coupler it is further understood that the coupler also prevents the user or others from touching and contaminating the needle or hollow spike. Also, the coupler itself can further have a removable cover or seal protecting the inside of the coupler and preventing access until the cover or seal is removed. The coupler can also have a similar cover of silicon or other material capable of being punctured for protecting the needle or hollow spike wherein the silicon or puncturable material is punctured by the needle during placement on the bacteria detection bottle.

One advantage of the present invention, as mentioned above, is that use of a sample bulb, container or sampler will isolate the product of the collection bags from further contamination.

The examples of the above described apparatus and methods are for illustrative purposes only. Variations will become apparent to those skilled in the art. Such variations and other modifications are included within the scope and intent of the invention.

We claim:

1. A pre-connected disposable for blood processing apparatus comprising:
a blood processing vessel;
a blood product container fluidly connected to the blood processing vessel;
a blood sampling container pre-connected to the blood product container;
a needle or hollow spike pre-connected to the blood sampling container wherein a sample of blood product can flow from the blood product container to the blood sampling container and to the needle or hollow spike.

2. The pre-connected disposable of claim 1 wherein the blood processing apparatus is an apheresis system for collecting selected separated blood components and wherein the blood processing vessel comprises a blood separation vessel.

3. The pre-connected disposable of claim 1 wherein the blood product container is a bag for collecting platelets.

4. The pre-connected disposable of claim 1 wherein the blood product container is a bag for collecting plasma.

5. The pre-connected disposable of claim 1 wherein the blood product container is a bag for collecting red blood cells.

6. The pre-connected disposable of claim 1 wherein the blood sampling container comprises a sample bulb.

7. The pre-connected disposable of claim 6 further comprising:
a coupler around the needle or hollow spike adapted for coupling to a bacteria detection device.

8. The pre-connected disposable of claim 7 wherein the coupler is adapted to provide an aseptic connection to a bacteria detection device.

9. The pre-connected disposable of claim 1 wherein the blood sampling container is a sample bag.

10. The pre-connected disposable of claim 9 further comprising a coupler around the needle or hollow spike adapted for coupling to a bacteria detection device.

11. The pre-connected disposable of claim 10 wherein the coupler is adapted to provide an aseptic connection to the bacteria detection device.

12. The pre-connected disposable of claim 1 further comprising:
a coupler around the needle or hollow spike adapted for coupling to a bacteria detection device.

13. The pre-connected disposable of claim 12 wherein the coupler is adapted to provide an aseptic connection to the bacteria detection device.

14. The pre-connected disposable of claim 1 further comprising:
a Y-connector between the blood sampling container and the needle or hollow spike for pre-connecting the needle or hollow spike to the blood sampling container.

15. The pre-connected disposable of claim 14 further comprising:
tubing between the Y-connector and the needle or hollow spike; and
a clamp on the tubing.

16. The pre-connected disposable of claim 1 further comprising:
tubing between the Y-connector and the needle or hollow spike; and
a frangible connector in the tubing.

17. The pre-connected disposable of claim 6 wherein the sample bulb further comprises:
a first sample bulb end;
a second sample bulb end;
wherein the sample bulb is pre-connected to the blood product container at one of the first or second sample bulb ends and wherein the sample bulb is pre-connected to the needle or hollow spike at the other of the first and second sample bulb ends.

18. A sample kit adapted to be attached to a biological fluid product container or a tissue or cell culture container and adapted to be coupled with a bacteria detection device comprising:
first tubing adapted to be sterilely connected to a biological fluid product or tissue or cell culture container;
a sample container pre-connected to the first tubing;
a needle or hollow spike pre-connected to the sample container and adapted to cooperate with a bacteria detection device.

19. The sample kit of claim 18 wherein the sample container comprises a bag.

20. The sample kit of claim 19 further comprising indicia indicative of fluid levels on the bag.

21. The sample kit of claim 18 further comprising:
a coupler around at least a portion of the needle or hollow spike and adapted to be coupled to a bacteria detection container.

22. The sample kit of claim 18 wherein the sample container comprises a sample bulb.

23. The sample kit of claim 22 further comprising second tubing pre-connected to the sample bulb wherein the sample bulb comprises:
a first sample bulb end; and
a second sample bulb end wherein the first sample bulb end is pre-connected to one of the first and second tubing and the second sample bulb end is pre-connected to the other of the first and second tubing.

24. The sample kit of claim 21 wherein the coupler permits the needle or hollow spike to be aseptically coupled to a bacteria detection device.

25. A method of detecting bacteria or microorganisms in a bodily fluid comprising:
removing a sample of the bodily fluid from a bodily fluid container to a sample container;
removing the bodily fluid container from the sample container to isolate the remaining bodily fluid in the bodily fluid container;
aseptically piercing a bacteria detection container with a needle or hollow spike; and
flowing the sample from the sample container through the needle or hollow spike to the bacteria detection container;
detecting any change of condition indicative of bacteria or microorganisms in the sample in the bacteria detection container.

26. The method of claim 25 further comprising:
sterilely connecting the sample container to the bodily fluid container.

27. The method of claim 25 further comprising:
sterilely connecting the needle or hollow spike to the sample container.

28. The method of claim 25 further comprising:
pre-connecting the sample container to the bodily fluid container.

29. The method of claim 28 further comprising:
pre-connecting the needle or hollow spike to the sample container.

30. The method of claim 25 further comprising:
pre-connecting the needle or hollow spike to the sample container.

31. The method of claim 25 wherein the removing step further comprises squeezing the sample container to receive the sample.

32. The method of claim 25 wherein the removing step further comprises flowing by gravity the sample from the bodily fluid container to the sample container.
